(19) United States
(12) Patent Application Publication
(10) Pub. No.: US 2003/0149406 A1
(43) Pub. Date: Aug. 7, 2003

(54) Multi-layer dressing as medical drug delivery system

(76) Inventors: Lucie Martineau, Kettleby (CA); Pang Nin Shek, Toronto (CA)

Correspondence Address: D.W. EGGINS, 18 DOWNSVIEW DRIVE, BARRIE, ON, L4M 4P8, CA

(21) Appl. No.: 10/358,165

(22) Filed: Feb. 5, 2003

Related U.S. Application Data
(60) Provisional application No. 60/354,288, filed on Feb. 7, 2002.

(51) Int. Cl. A61F 13/00

(52) U.S. Cl. 604/304; 604/360; 604/368

(57) ABSTRACT

A medicated multi-layered polyurethane foam dressing and drug delivery device with cooling properties, used in body cavities and on damaged tissues, particularly bums. The dressing may have at least one therapeutic agent dispersed into the polyurethane layers; an optional outer layer of either a hydrogel formulated from a polyurethane or an adhesive elastomeric material; an optional drug-free fluid-retaining layer and/or a drug-reservoir layer, each comprised of hydrophilic polyurethane foam; a non-adherent, drug-loaded surface-contacting layer of a polyurethane hydrogel; and an optional cover sheet, for surface dressings. An interposed liquid transfer control may be used at a layer interface. The conformable dressing shapes include cylindrical, oval, flatsheets in pre-determined sizes; and may be secured with a secondary dressing. The contact surface may be channeled to enhance fluid distribution.

Cross-Reference To Related Applications Provisional Application No. 60/354,288 filed Feb. 7, 2002.

### STATEMENT REGARDING FEDERALLY SPONSORED RESEARCH OR DEVELOPMENT

Not Applicable (N/A).

### REFERENCE TO A SEQUENCE LISTING, A TABLE, OR A COMPUTER PROGRAM LISTING

(N/A)

### COMPACT DISC APPENDIX

N/A

### BACKGROUND OF THE INVENTION

1. This invention is directed to a dressing for personal use, and in particular to a mass-produced, polymer-based, multi-layer dressing that may include a drug or therapeutic agent delivery system.

2. Historically, a wide range of treatments and dressings have evolved for dealing with surface skin conditions, as well as with flesh injuries and wounds. As an instance, wound care forms an important aspect of post-operative surgery, and can play an integral role in wound healing.

Increased understanding of the healing process has led to a wide range of improvements in wound care products, particularly in wound dressings. Much of this prior art is listed in the attached Information Disclosure Statement, and commented upon therein. To summarize, it is the opinion the present inventors that no single one of the cited prior art deals satisfactorily with all of the physiological requirements that underlie optimal wound healing.

It is asserted herein that an ideal wound dressing should be absorbent; be minimally adherent to the wound bed, to reduce the risk of re-injury upon removal of the dressing; have a therapeutic activity (e.g., analgesic, bactericidal, hemostatic, etc.); and, exert a soothing and/or cooling effect upon application to a wound, especially a bum wound.

It is understood that there is presently no commercially available dressing that possesses all of the above characteristics, nor is such taught in the Prior Art listed in the attached Information Disclosure Statement, that forms a part of this application.

### BRIEF SUMMARY OF THE INVENTION

This invention is directed to a medicated, absorbent, minimally adherent dressing (including wound dressings), in a polymer-based platform. More specifically, the present invention relates to a polyurethane therapeutic agent delivery device made up of at least two layers, each layer containing at least one agent, such as a drug, of a single concentration or in a combination of concentrations. The dressing comprises one layer of hydrophilic polyurethane foam, preferably HYPOL™ polyurethane, and at least one surface-contacting layer of hydrogel, preferably HYPOL hydrogel. The hydrophilic layer functions as a drug reservoir capable of absorbing the excess exudate, while the hydrogel layer acts as a minimally adherent surface that maintains the wound bed adequately moist for optimal wound healing and exerts a soothing cooling effect. The multi-layered agent delivery device has been found to be useful in cooling the surface to which it is applied; in preventing or alleviating bacterial contamination of wounds, as demonstrated in several animal models; and, in serving as a vehicle for delivering an analgesic agent.

Hydrogels are important wound care products, with a unique ability to maintain the wound bed moist and to cool the surface on which they are applied. However, a distinct disadvantage of commercially available hydrogel wound dressings is that they do not provide a barrier against wound infection. There do not appear to exist any commercially available medicated hydrogel wound dressing sheets. While it is often recommended clinically that an antimicrobial agent be applied under a hydrogel dressing, or that it be blended with an amorphous hydrogel, which could provide some control of bacterial growth, it is frequently impractical to do so, as it constitutes a further step in wound care management. Therapeutic substances have been added to gel pads or bandages to provide additional bacterial control and other therapeutic effects. Examples of medicated hydrogel products are disclosed in the referenced Information Disclosure Statement. However, hydrogel wound dressings have typically a relatively high water content (>90%), which significantly restricts their absorbency capacity, such that a secondary dressing is usually required to absorb the excess wound exudate.

Commercially available polyurethane wound dressings are therefore important wound care products, especially since they can absorb moderate to high volumes of wound exudates. Examples of mono-layer polyurethane products are disclosed in the referenced Information Disclosure Statement. However, none of the listed prior art discloses of incorporating drugs or other agents into the HYPOL foam or HYPOL hydrogel, nor is there a disclosure of having a multi-layer wound dressing.

While there are no known, presently available commercial medicated hydrophilic polyurethane wound dressings, the patent literature is replete with references to multilayer polyurethane wound dressings (preferably made of HYPOL polyurethane foam) in which therapeutic substances may be incorporated. However, there is no disclosure in the prior art that the layers of the multi-layered polyurethane wound dressing comprise HYPOL of different physico-chemical characteristics as disclosed herein. Furthermore, the referenced prior art does not disclose incorporating at least one active agent in each of the hydrophilic polyurethane layers so that such active agent or drug may be in different concentration in each of the layers.

In a preferred embodiment of the wound dressing system of the present invention, the first primary layer is preferably composed of HYPOL polyurethane foam pre-polymer, or another similar hydrophilic polyurethane pre-polymer (from hereforth, hydrophilic polyurethane pre-polymers will be referred to generically as HYPOL), and serves as a drug-containing layer or drug reservoir to hold at least one drug (such as an antibacterial agent, analgesic, or clotting agent, etc.). The second primary layer is a drug-loaded, minimally adherent surface-contacting layer, and may be composed of HYPOL hydrogel (or any other suitable hydrogel).

In another dressing embodiment, a HYPOL drug-reservoir layer can be sandwiched between two surface-contacting or “face” layers of HYPOL hydrogel (or any other suitable hydrogel), for use as a packing material in deep wounds or body cavities.

In yet another embodiment, the HYPOL drug-reservoir layer can be sandwiched between one drug-free highly hydrophilic polyurethane foam layer and one surface-contacting layer of HYPOL hydrogel, for use as a dressing device in highly exudating wounds. Each of the layers of HYPOL (i.e., surface-contacting and drug-reservoir layers) in the dressing device may have different physico-chemical characteristics.

The provisions for the physico-chemical characteristics of different HYPOL layers is determined by the functional role of each layer. For example, the surface-contacting HYPOL hydrogel layer has an elevated water content to promote cooling upon its being applied to a surface of a host such as a vertebrate host, and to reduce adhesion of the dressing to the wound surface. The drug-reservoir layer has a physicochemical composition, made up of HYPOL and possibly other blending agents, that favors the release of the drugs incorporated therein. Furthermore, a HYPOL layer that serves the primary function of removing and retaining wound exudate fluid requires a physico-chemical characteristic that promotes moisture retention.

It will be understood that the positioning, in mutually adhering relation, of two surfaces having markedly different hydration levels, such as the hydrophilic HYPOL layer and the hydrogel layer in the subject dressing, may cause a transfer of moisture from one layer to the other. This potential moisture transfer can be addressed by modifying appropriately the physico-chemical composition of either layer.

In yet another preferred embodiment, a self-regulating, flow-sensitive polymeric or synthetic membrane is placed between the hydrophilic HYPOL layer and the hydrogel layer with the intent that the membrane prevents passive moisture transfer from the hydrogel layer to the HYPOL layer, while the presence of a moderate to high flow of exudate triggers the physical modification of the membrane to facilitate moisture transfer to the drug-reservoir HYPOL layer.

In a preferred embodiment of the dressing device of the present invention, the use of HYPOL polyurethane foams with different physico-chemical characteristics enables the use of a chemical process intrinsic to polyurethane foams to cure the two layers together. However, the use of existing known processes for laminating such layers together, such as heat sealing; welding by radio frequency welding; ultrasonic welding; or adhesives is also contemplated.

The surface-contacting layer of a dressing embodiment may incorporate at least one drug. The entire drug delivery system of the dressing may include at least two or more different drugs. The same drug can be incorporated in both a drug-reservoir layer and the surface-contacting layer(s), or different drugs can be incorporated in the surface-contacting face layer(s) and in another, drug retention reservoir layer. If the same drug is incorporated in the drug delivery system of the dressing, the concentration of that drug in each of the two layers may be different.

In a method of use aspect, the present invention provides a method of administering to a wound in a predetermined, controlled manner at least one therapeutic agent, by applying to the wound a dressing product of this invention for an extended period of time. It will be understood that the subject dressing may contain different concentrations of the same agent in the each of the layers.

In another aspect, the subject method of administering in a slow, sustained manner at least one therapeutic agent to an intact surface of a vertebrate host comprises inserting an appropriate dressing product of this invention for an extended period of time in a natural body cavity of the host. It will be understood that the subject dressing may contain different concentrations of the same agent in the each of the layers.

Another aspect of this method, for treating external wounds, uses a hydrophilic polyurethane foam (preferably HYPOL polyurethane) as a dressing device having two layers, with at least one active therapeutic agent present in each of the layers. If the same drug is incorporated in each of the layers, then the concentration of that drug in each of the layers may be different.

Yet another object of this invention is to provide a method for treating external wounds using a hydrophilic polyurethane foam (preferably HYPOL polyurethane) dressing device that has a surface-contacting layer that will rapidly release at least one therapeutic drug, and a reservoir layer that will thereafter slowly release at least one therapeutic agent over an extended period of time, preferably for up to 15 days.

The safe time of effectiveness over which a drug delivery dressing may remain inserted in a body cavity or wound varies in accordance with the condition of the subject and the immediate condition of the site.

The present invention includes methods for making the subject medicated multi-layered polyurethane drug-delivery dressing.

The present method provides the capability for incorporating at least one different drug into each layer of the subject multi-layer drug delivery dressing.

Yet another embodiment provides a method for incorporating at least two different drugs into each of the layers of a single or of a subject multi-layer drug delivery dressing.

The use of HYPOL layers having different physico-chemical characteristics, as presently disclosed, in the subject wound dressing, is novel; as does the present provision of a multi-layered wound dressing that can 1) incorporate a combination of therapeutically active components in the appropriate layers; 2) has the capacity to handle a wide range of wound exudate volumes from a given wound; and 3) includes a minimally adherent surface-contacting layer that can also provide cooling to the surface to which it is applied.

It is intended to cover all alternatives, modifications and equivalents as may be included within the spirit and scope of the invention as defined by the appended claims. As an example, while the present invention will be described herein as primarily useful as a surgical or first-aid wound dressing, it should be appreciated that the foam composition with similar characteristics could also be used for cosmetic applications.

### BRIEF DESCRIPTION OF THE SEVERAL VIEWS OF THE DRAWINGS

Certain embodiments of the invention are described by way of illustration, without limitation of the invention thereto, other than as set forth in the present claims, reference being made to the accompanying drawings, wherein:

FIG. 1A is a perspective view of a drug delivery dressing having two drug-loaded polyurethane layers, as a first embodiment of the present invention;

FIG. 1B is a cross-sectional view of a portion of the FIG. 1A embodiment;

FIG. 2A is a perspective view of a three-layer, drug-loaded embodiment;

FIG. 2B is a cross-sectional view of a portion of the FIG. 2A embodiment;

FIG. 3A is a perspective view of a multi-layer embodiment incorporating a protective release sheet;

FIG. 3B is a cross-sectional view of a portion of the FIG. 3A embodiment;

FIG. 4A is a perspective view of a drug delivery dressing having three polyurethane layers, only one of which being drug-free, as a fourth embodiment of the present invention;

FIG. 4B is a cross-sectional view of a portion of the FIG. 4A three-layer embodiment;

FIG. 5 is a graphical representation of the effectiveness of chlorhexidine-loaded dressings of the present invention, simultaneously loaded or not with the analgesic fentanyl citrate, in preventing the spread of infection in superficial and deep tissues underlying full-thickness wounds;

FIG. 6 is a graphical representation similar to that illustrated in FIG. 5, using cerium nitrate as the antiseptic agent in the subject dressing;

FIG. 7 is a graphical representation comparing the effectiveness of chlorhexidine-loaded dressings of the present invention in preventing the spread of infection in superficial and deep tissues underlying full-thickness wounds to that of a commercial chlorhexidine-loaded dressing;

FIG. 8 is a histogram illustrating the effects of shelf-life on reducing the in vitro bactericidal efficacy of the chlorhexidine-loaded dressings of the present invention.

FIG. 9 is a graphical representation comparing the cooling efficacy of the subject dressing to that of commercial dressings.

### DETAILED DESCRIPTION OF THE INVENTION

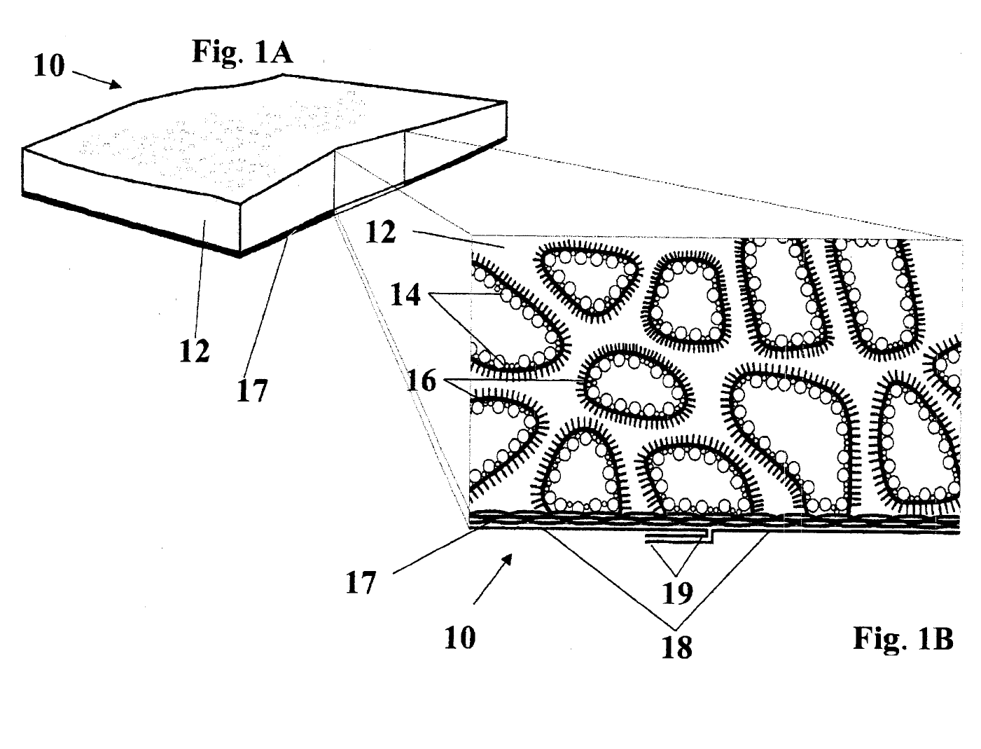

Referring to FIGS. 1A and 1B, a dressing 10, being a first embodiment of the present invention, has a layer 12 of polyurethane foam, preferably the aforementioned HYPOL, constituting a reservoir for a selected drug or other therapeutic agent, represented by the elements 14 and 16. The layer 12 is adhered to a hydrogel polymer layer 17, which may contain at least one drug (not shown). The outer (lower) face of the layer 17 is protected by a two-piece cover sheet 18, well known in the art, and having a pair of pull-tabs 19, to facilitate removal of the cover sheet 18. In use, the protective cover sheet 18 is removed from the dressing 10, and the outer hydrogel layer 17 is applied to the injured surface. The cover sheet 18 preserves the sterility of the drug delivery device, and sustains the hydration of the hydrogel layer 17. Used as a surface dressing, the dressing 10 may be secured in place by way of a secondary dressing such as a bandage, tubular dressing, etc. It will be understood that the dressing 10 may be of cylindrical shape, for use as a packing within a deep wound, or a body cavity, where a fastening means is not usually required. The first embodiment of the subject dressing may also be used as a containment device for spilled internal organs.

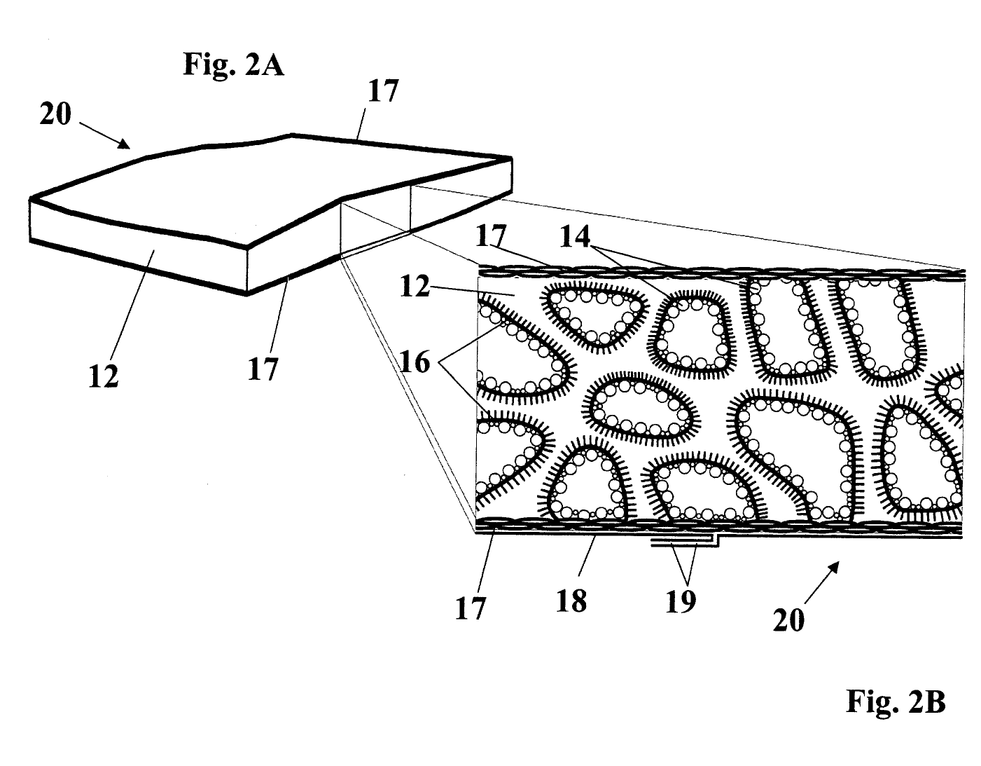

Referring to FIGS. 2A and 2B, a dressing 20, being a second embodiment of the present invention, has a drug-reservoir layer 12 comprised of a hydrophilic polyurethane foam layer, being illustrated as having two drugs 14, 16 in dispersed relation therein; the layer 12 being cast, as disclosed herein, over a surface-contacting hydrogel polymer layer 17, which may contain at least one drug therein (not shown), and is protected by a cover sheet 18. A second hydrogel polymer layer 17, which also may contain at least one drug therein, is cast in adhering relation on top of the drug reservoir layer 12. This top layer 17 also may be protected by a cover sheet 18 (not shown). In use, the second embodiment multi-layered drug delivery device 20 can be used as a packing material for peritoneal wounds after removal of both portions of the cover sheet 18, and thus does not require further means of attachment to the patient other than what is dictated by conventional abdominal surgical procedures.

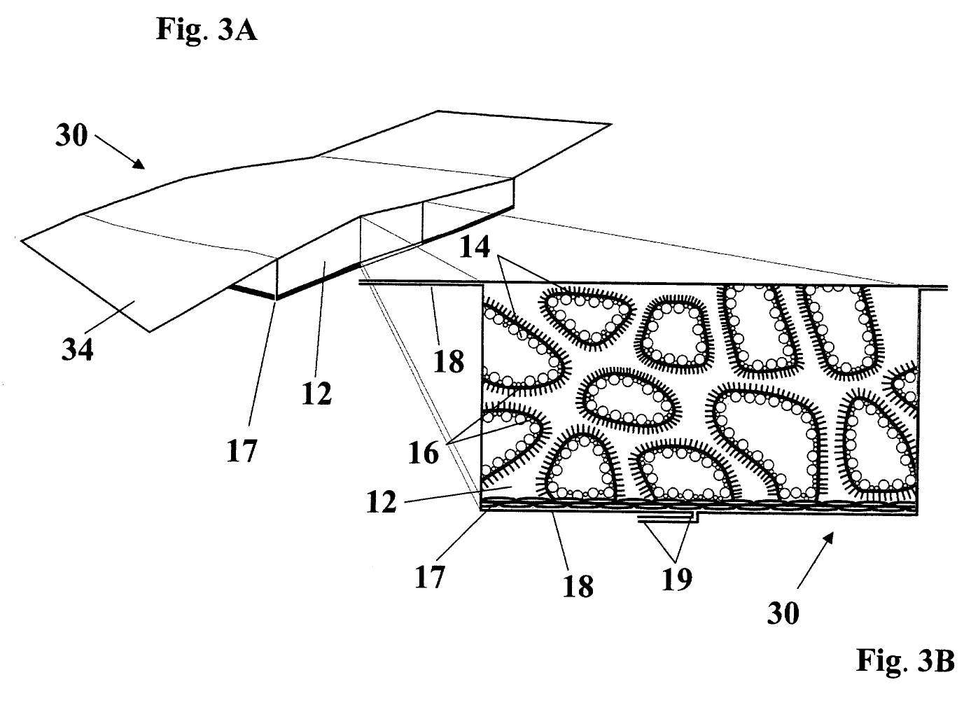

Referring to FIGS. 3A and 3B, in this third embodiment of the invention, a dressing 30 has a drug-reservoir layer 12 comprised of a hydrophilic polyurethane foam layer being illustrated as having two drugs 14, 16 in dispersed relation therein, is cast as disclosed herein over a wound surface-contacting layer 17 comprised of a hydrogel polymer, the layer 17 also containing at least one drug (not shown). An adhesive outer elastomeric layer 34 extending beyond the edges of the layer 12, providing a means of attachment to the patient, completes the dressing 30 as a bi-layer drug-delivery device. The two adhesive under-surfaces of the layer 34 and the hydrogel layer 17 are each protected by a respective cover sheet 18. In use, the third embodiment multi-layered drug delivery device 30 can be used as a band-aid for superficial wounds or as a compression bandage for hemorrhagic penetrating wounds.

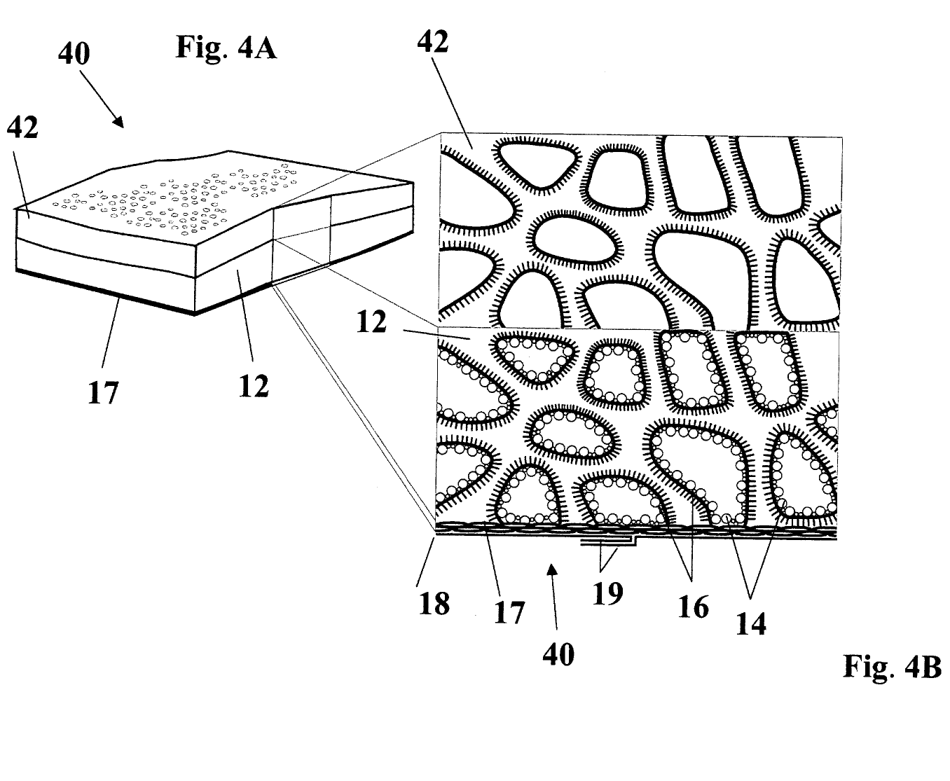

Referring to FIGS. 4A and 4B, a tri-layer dressing 40 has a drug-free, highly hydrophilic polyurethane foam layer 42; a drug-reservoir layer 12 comprised of a hydrophilic polyurethane foam layer in which at least one drug 14 is dispersed; a surface-contacting layer 17, comprised of a hydrogel polymer, the latter also containing at least one drug 14 (not shown). The tri-layer drug delivery dressing 40 may be secured in place using a secondary dressing, or combined with an adhesive outer layer 34 (not shown). By tailoring the reactant mixture formulation of the drug-free foam layer 42 of the dressing 40, it can also be used as a compression bandage for heavily hemorrhagic penetrating wounds.

Concerning the make-up and fabrication of the subject dressings, they can be manufactured to assume various shapes (e.g., cylindrical, oval, islands, etc.) or flat sheets in various pre-determined sizes. Preferably, the dressings are prepared under aseptic conditions, packaged in aluminum foil laminated bags with a heat-sealable film, and sterilized in the package. Favoured procedure is by gamma sterilization. Alternatively, the dressing can be sterilized by ethylene oxide and heat sterilization.

The preferred composition of the drug-free fluid-retaining layer, the drug-reservoir layer, and the surface-contacting layer for the drug delivery dressing of the present invention are described in detail herein. The term hydrophilic polymer foam as used herein means any foam that will absorb fluids such as water, blood, wound exudates (including blister fluid) and other body fluids (including peritoneal fluid).

Favored hydrophilic polymer foams are hydrophilic polyurethane foams. HYPOL pre-polymer foams form a preferred group of foams within the general description of hydrophilic polymer foams. HYPOL foams can be made from HYPOL hydrophilic isocyanate terminated pre-polymers marketed by DOW Chemicals. Ideally, the hydrophilic foam will absorb at least four times its weight of fluids. Suitable foams may be prepared from hydrophilic materials per se or may be treated to render them hydrophilic (e.g., with surfactants, super-absorbent agents, etc.). However, it is preferable that the foam be hydrophilic per se, since the incorporation of chemicals (including drugs, surfactants, etc.) may alter the physical characteristics (e.g., absorption, porosity, pore size, etc.) of the foam material. It is also desirable that the hydrophilic polymer foam layer absorbs the wound exudate rapidly as this prevents undesirable maceration of the wound by the accumulation of exudates beneath or at the face of the dressing. The hydrophilic foam should also be conformable (i.e., soft and compressible, not stiff or rigid), so that the hydrophilic foam when placed in a body cavity will conform readily to the contours of the wounds, whether the patient is resting or moving.

The type and amount of prepolymer in the reactant mixture used to prepare a hydrophilic foam layer will depend on a number of factors, including the proportion of other components in the reactant mixture.

In a preferred embodiment of the layer composition, the drug reservoir layer 12 will be made of HYPOL 2002, while the wound-contacting layer 16 will be made of a mixture of HYPOL 50G and 2060G. In all instances, there should be sufficient prepolymer and water to form a polyurethane foam or hydrogel layer of suitable thickness, so as to contain therapeutic levels of the drugs selected. There should also be sufficient prepolymer to provide mechanical integrity to each of the layer compositions, but not too much, so that the resulting compositions become unworkable.

The reactant mixtures of each layer of the present invention may further include an adjuvant to extend the curing time of the foam or hydrogel reactant mixture, thereby allowing a thorough mixing of the mixtures prior to spreading them sequentially into layers of suitable thickness for curing. Preferably, the adjuvant selected is water-soluble and biocompatible (i.e., does not exert harmful effects upon contacting the wound bed or skin). It is also preferable that the selected adjuvant be compatible with the pre-polymers selected as well as with the therapeutic agents or other additives incorporated into the reactant mixtures. Suitable adjuvants include water-soluble alcohols, including monols, diols, and polyhydric alcohols. Preferably, the reactant foam or hydrogel mixtures should contain less than 0.01% of alcohol by weight.

The drug delivery dressing of the present invention preferably contains at least one physiologically active agent that is released at the face of the dressing to the contact site (e.g. wound/intact skin) in therapeutically effective amounts. The drug delivery dressing may contain a drug or combination thereof selected from a group including but not limited to: broad spectrum antibiotics, antimicrobials, antifingals, antipathogenic peptides, antiseptics, hemostatic agents, local analgesics, central nervous acting agents, wound healing agents (e.g., growth factors), immunosuppressives, and all safe drugs that can be delivered to human tissues. At least one drug may be contained in each of the drug-reservoir layers and the surface-contacting face layer. If the same drug is contained in both layers, then the layers may contain two different concentrations of the drug.

Each drug selected should be chemically compatible with the additional components of each of the reactant mixtures. Furthermore, when more than one therapeutic agent is incorporated, all the drugs selected should be deemed chemically compatible prior to their incorporation in the dressing. It should be appreciated by those skilled in the art that the amount of each of the therapeutic agents incorporated in the wound dressing of the present invention can be varied, depending on the agent, the intended dosage, the individual undergoing treatment, the particular condition indications and the like.

Those skilled in the art will appreciate that the concentration of the drug incorporated in each layer is a function of both the intrinsic activity of the therapeutic agent as well as the drug-release characteristics of the chemical formulation of the HYPOL layer prepolymer. The dose range of the therapeutic agents can be determined by animal wound modeling studies.

The physiologically active agents may be incorporated during the process of manufacturing the drug delivery device. In a preferred embodiment, aqueous solutions of the selected free drugs are prepared, and used as part of the reactant mixtures to prepare each of the layers (i.e., drug reservoir and surface-contacting layers). The layers are then cast in sequence as described herein. This method is preferred either when different drugs or different concentrations of a given drug are to be incorporated into the respective layers.

In yet another embodiment of drug loading, the drugs may be entrapped in a delivery system such as liposomes, microspheres, and the like, to further extend the drug release characteristics to the drug delivery device, and incorporated in the reactant mixtures to prepare each of the layers.

In another subject method of drug loading, the therapeutic agents may be incorporated after the dressing layers have cured and the multi-layer drug delivery device is made, by immersing the subject drug delivery device in an aqueous solution containing the selected drugs. The drug delivery layer or dressing is then compressed during the immersion to expel any entrapped air. When fully saturated with the solution, the drug delivery layer is removed, and again compressed to a predetermined extent to expel any excess drug solution.

In an alternative method of drug loading, the drug delivery devices of the present invention are immersed in an aqueous solution containing therapeutic levels of the drugs selected. The immersed dressings are then placed into a hyperbaric chamber for a specific period of time in which the entrapped air becomes expelled and the drugs are forced into the dressings. After completion of the drug loading procedures, the drug delivery devices are removed from the pressure chamber, and mechanically compressed to a predetermined extent to expel any excess drug solution.

Drug delivery dressings prepared by the foregoing methods, using various aqueous concentrations of chlorhexidine, were found to demonstrate significant in vitro and in vivo bactericidal properties.

While the surface-contacting hydrogel layer of the present invention preferably contains only the cited pre-polymers, water, adjuvant, and at least one therapeutic agent, the drug-reservoir layer may contain a number of other chemicals described in detail herein, to further improve its hydrophilic properties. Thus, the foam reactant composition may include a hydrophilic agent that is incorporated into the foam mixture to absorb liquid (e.g. wound exudate, peritoneal fluid). The hydrophilic agent is preferably a highly absorbent polymer, commonly known as a super-absorbent polymer. The inclusion of such agent will increase the capacity of the wound dressing to tightly hold at least three times its weight in fluid after compression. Other potential additives could include polymers such as chitosan, alginate, etc., to improve the hydrophilic action of the HYPOL prepolymer.

The amount and type of hydrophilic agent used in the wound dressing will depend on the intended application of the invention. For example, for an ulcerating wound with large fluid exudate volume (e.g., a burn or a bleeding wound), a hydrophilic agent with a high uptake is desirable. On the other hand, for a laceration or abrasion, it may be more suitable to use a less hydrophilic agent or to use an agent with a lower fluid uptake. One skilled in the art can readily determine the type and amount of hydrophilic agent to be used.

The reactant foam mixture of the present invention may further include surfactants. Suitable and preferred biocompatible surfactants forming conformable hydrophilic polymer foams include non-ionic surfactants, such as oxypropylene oxyethylene block co-polymers known as Pluronics™ marketed by BASF Wyandotte, preferably Pluronic F68. Generally but not necessarily, the amount of surfactant should be up to 10% by weight of the foam reactant mixture. The selected surfactant should not react with the pre-polymer selected or any component of the reactant mixture to impair foam formation or to adversely affect the desired characteristics of the foam composition in use or while being stored. One skilled in the art can readily determine the type and amount of surfactant to be used.

The present invention also includes a method of manufacture of the drug delivery device, comprising the steps of mixing the appropriate reactants of the surface-contacting layer together in an appropriate receptacle to form a standardized aerated mix. The mixture is then spread at room temperature onto a smooth support to which it is not adherent (e.g., glass surface) to form a wound surface-contacting layer of predetermined thickness. The spreading may be effected by means of a spreader bar that is drawn over the surface of the mix at a fixed distance above it. The second layer (i.e., the drug-reservoir layer) is simultaneously prepared in the same manner, and applied to the wound-contact layer before the layers are fully cured.

For another embodiment, the mixture of the surface-contacting hydrogel layer is spread as described herein over a fully cured drug reservoir foam layer.

For yet another embodiment, a third layer comprising HYPOL hydrogel is cast on top of the drug reservoir layer already in adhering relation to another surface-contacting hydrogel layer.

In a further embodiment, the cured drug reservoir layer is immersed into the mixture containing the appropriate reactants of the surface-contacting hydrogel layer.

For yet another embodiment, the method of manufacture includes a second drug-free layer of HYPOL polyurethane being cast on top of the HYPOL drug-reservoir layer already in adhering relation to the surface-contacting hydrogel layer.

In yet another present method of manufacture, all layers are prepared, cast, and spread individually, and then sealed together using known methods of lamination (e.g., heat sealing, radio frequency welding, discontinuous adhesive, ultrasonic welding). It is further desirable that the pre-polymers selected be capable of curing in the absence of catalysts and at ambient temperature.

After preparing the drug delivery dressing embodiments of the present invention as described therein, the surface-contacting layer may be perforated or sliced through its thickness in several sites to create channels to enhance absorption of exudates.

For yet another embodiment, the mixture of the surface-contacting layer is sprayed over a fully cured drug reservoir foam layer to form a discontinuous hydrogel layer, thus enhancing the absorption of exudates into the hydrophilic polyurethane layer.

After curing, the surface-contacting hydrophilic hydrogel layer may have a thickness of up to 2.54 mm, preferably in the range 0.76 to 1.27 mm. After curing, the drug reservoir hydrophilic polymer foam layer may have a thickness of up to 10 mm, preferably in the range 3 to 7 mm. It will be appreciated by those skilled in the art that the thickness of the layers will depend, however, on a variety of considerations, including the quantity of each drug to be incorporated in each of the layers, the level of absorbency required, etc.

The following examples show by way of illustration and not by way of limitation, the practice of the present invention. These examples present data showing the in vitro and in vivo bactericidal activities as well as cooling efficacy of the drug delivery devices of the present invention.

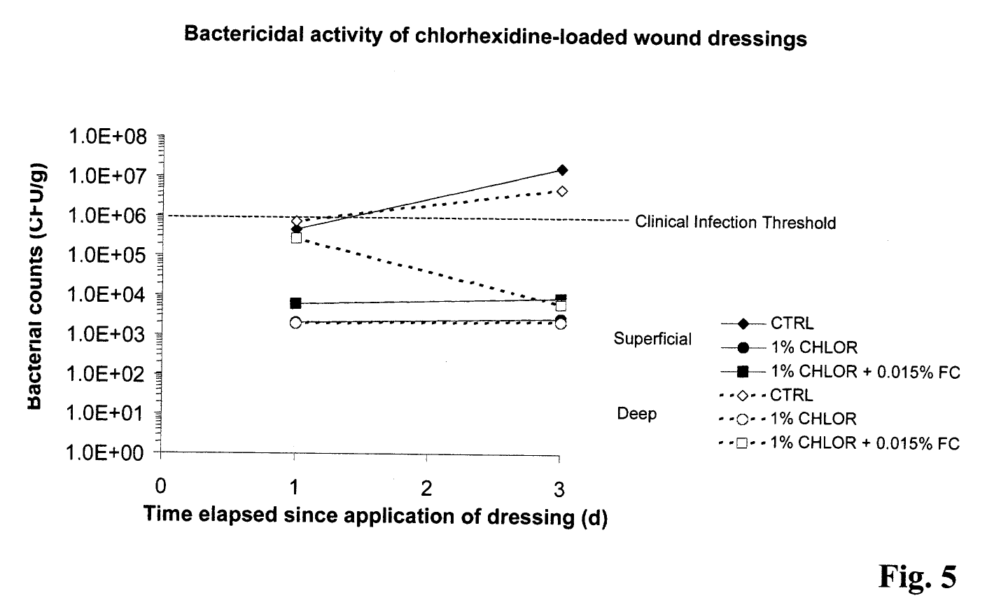

Referring to FIG. 5, the effectiveness of chlorhexidine-loaded wound dressing (CHLOR) in preventing the spread of infection in superficial (represented graphically in solid line) and deep tissues (shown in dashed line) was measured in rats with full thickness wounds. Wounds were covered for 24 hours or 72 hours with a drug-free wound dressing (shown graphically as diamonds), or with a dressing containing 1% chlorhexidine alone (“circles”) or in combination with 0.015% fentanyl citrate (“squares”). Data values are mean values±SEM (standard error of the mean; SEM) with a sample size (n) of three animals per experimental group.

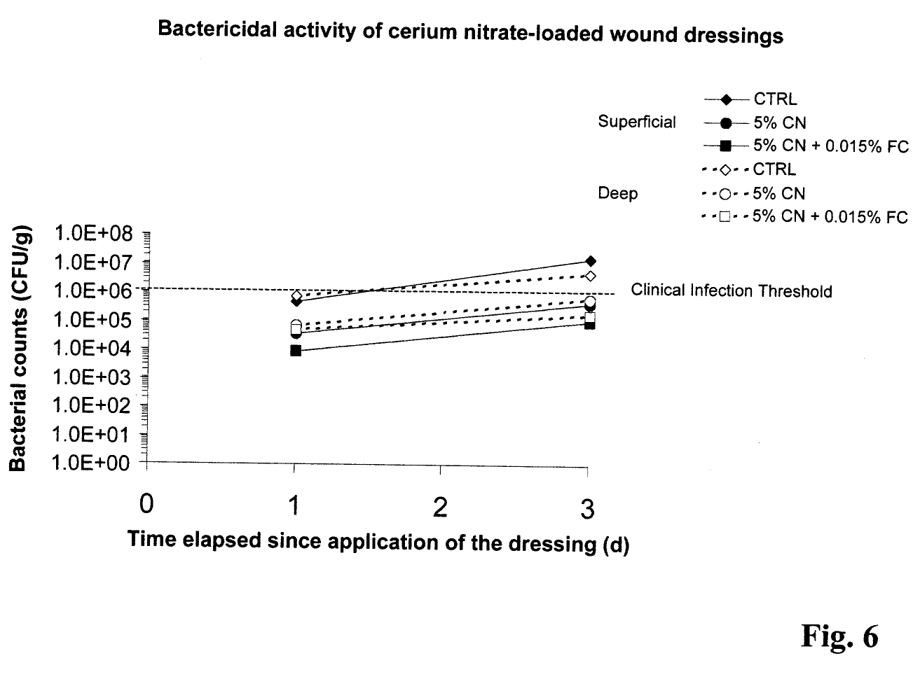

Referring to FIG. 6, the effectiveness of cerium nitrate-loaded wound dressings (CN) in preventing the spread of infection in superficial (represented graphically in solid line) and deep tissues (shown in dashed line) was tested in rats with full thickness wounds. Wounds were covered for 24 hours or 72 hours with a drug-free wound dressing (shown graphically as diamonds), or with a dressing containing 5% cerium nitrate alone (“circles”) or in combination with 0.015% fentanyl citrate (“squares”). Data values are mean values±SEM (standard error of the mean; SEM) with a sample size (n) of three animals per experimental group.

Those skilled in the art will appreciate that although chlorhexidine, cerium nitrate, and fentanyl citrate are the only drugs exemplified, many other therapeutic agents may also be successfully incorporated in each of the different wound dressing layers to exert therapeutic effects.

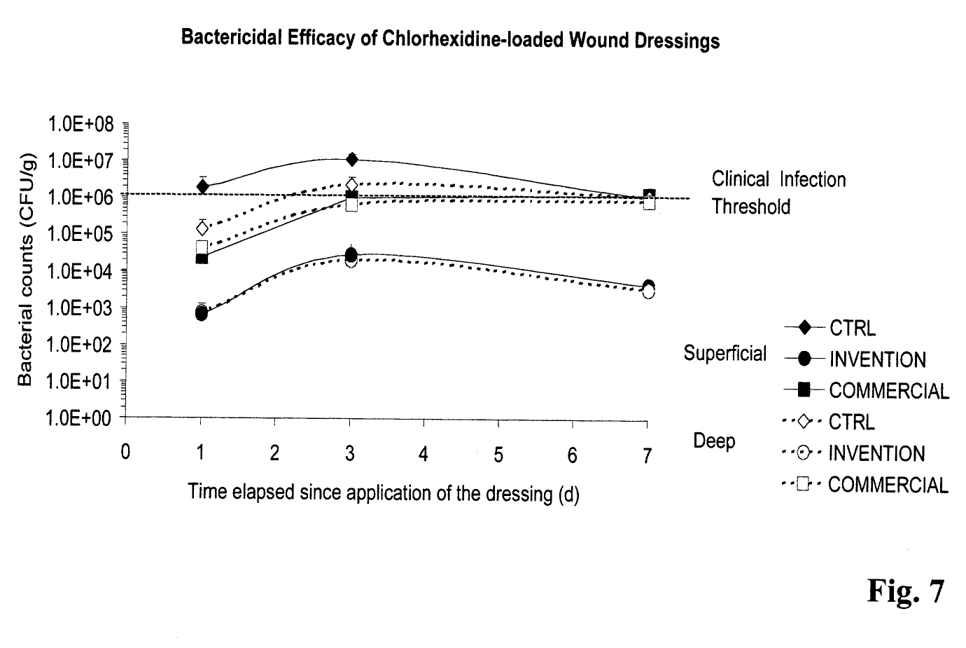

Referring to FIG. 7, this illustrates the effectiveness of chlorhexidine-loaded wound dressings in preventing the spread of infection in superficial (solid line) and deep tissues (dashed line) in rats with fill thickness wounds. Wounds were covered for 1, 3, or 8 days with a commercially available wound dressing containing chlorhexidine (“COMMERCIAL”—squares) or a dressing of the present invention containing no drug (diamonds) or comparable chlorhexidine levels (circles). Data values are means±SEM (n=6 per experimental group).

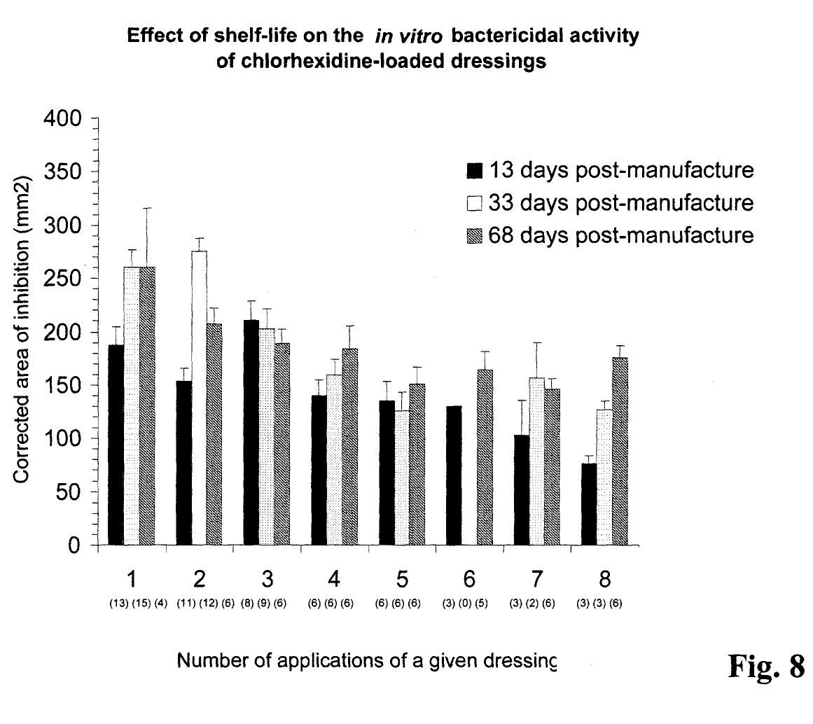

In the FIG. 8 histogram is illustrated the effect of shelf-life on the in vitro bactericidal efficacy of wound dressings of the present invention loaded with 1% chlorhexidine. The number of dressings tested in each experimental group is indicated in parenthesis. Data values are mean values±SEM.

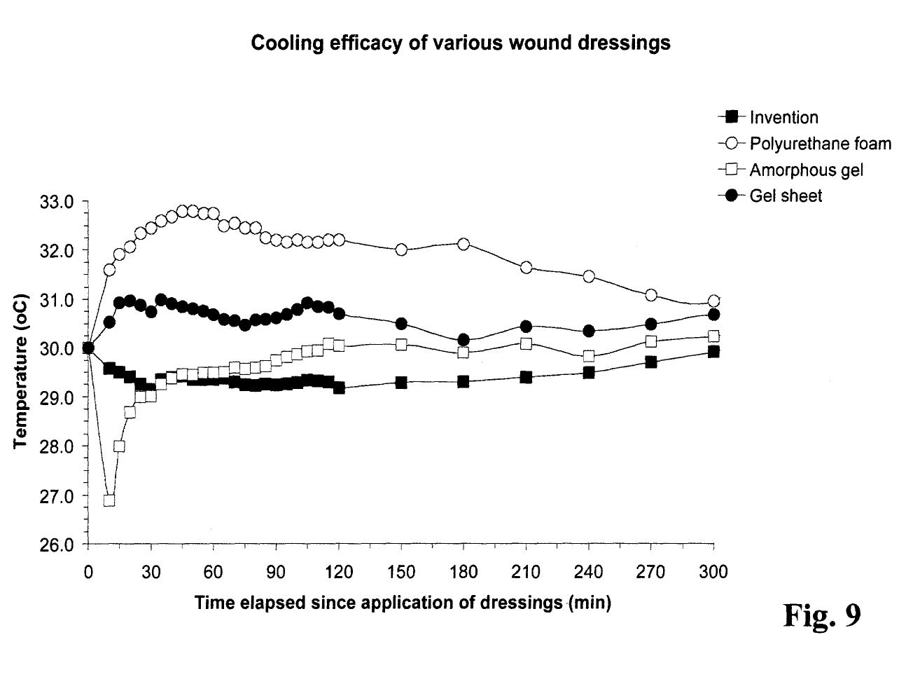

Referring to FIG. 9, this illustrates the effectiveness of various wound dressings in cooling human skin (n=8). The skin temperature of the subjects upper arms was monitored for 6 hours, when covered respectively with a drug-free wound dressing of the present invention (diamonds); a commercial hydrogel sheet (circles); a commercial polyurethane foam dressing (triangles); or a commercial amorphous gel (squares).

### EXAMPLE 1

### Reference: FIG. 5

This study was designed to simulate a scenario where a ‘fresh’ full-thickness wound was contaminated with bacteria. However, cleaning or debriding of the wound could not be performed immediately. Under those circumstances, first aid treatment consisted of applying a medicated wound dressing of the present invention to attempt to limit the progression of the superficial infection to deeper tissues, and to provide immediate analgesic relief.

Rats were anesthetized and two full-thickness wounds were made on the lateral side of their abdomen. A sterile gauze was inserted into each wound, and wetted with approximately 10⁹ Colony Forming Units (CFU; in 500 μL) of a clinical strain of Pseudomonas aeruginosa. A medicated dressing of the present invention, containing either 1% chlorhexidine (n=4) or 1% chlorhexidine with 0.015% fentanyl citrate (n=4), was applied to cover the entire wound. Six similarly wounded rats received a control dressing (i.e., drug-free).

The medicated dressings were prepared by immersing drug-free dressings in an aqueous solution of the drug(s) and then exposing the dressings to hyperbaric pressure (140 PSI) for 3 hours. A dressing was then secured to each rat. All animals were humanely sacrificed 24 hours or 72 hours after application of the experimental dressing, and muscle tissue samples were excised. Bacterial content was assessed in part of the tissues using standard microbiological procedures, while the remaining tissues were preserved for subsequent determination of the levels of the analgesic agent.

All wound dressings recovered were intact and were removed easily from the wound bed (i.e., having no adherence). Assessment of bacterial counts in the panniculus carnosus (i.e., superficial muscle) and the external oblique muscles (i.e., deep muscle) revealed approximately 3-log reductions in the number of bacteria recovered in these tissues after application of the chlorhexidine-loaded dressings for 24 hours compared to that of the control dressings (FIG. 5). Comparable reductions were observed in the panniculus carnosus and the external oblique muscles exposed to both chlorhexidine and fentanyl citrate. These reductions in tissue contamination were maintained for 72 hours, while the untreated wounds became further infected.

These data suggest that application of chlorhexidine-loaded or chlorhexidine/fentanyl citrate-loaded wound dressings of the present invention for up to 72 hours is an effective way for preventing the progression of infection in contaminated wounds.

### EXAMPLE 2

### Reference: FIG. 6

Rat wounds were infected as previously described in EXAMPLE 1. A medicated dressing of the present invention dressing, containing either 5% cerium nitrate (n=3) or 5% cerium nitrate with 0.015% fentanyl citrate (n=3), was applied to cover the entire wound. Six other animals received a control dressing (i.e., drug-free). Remaining experimental procedures were as described in EXAMPLE 1.

All wound dressings recovered were intact and were removed easily from the wound bed (i.e., no adherence). Assessment of bacterial counts in the panniculus carnosus (i.e., superficial muscle) and the external oblique muscles (i.e., deep muscle) revealed approximately 1.5-log reductions in the number of bacteria recovered in these tissues after application of the cerium nitrate-loaded dressings for 24 hours compared to that of the control dressings (see FIG. 6). Comparable reductions (approximately 2.0 log) were observed in the tissues exposed to both cerium nitrate and fentanyl citrate. While all experimental wounds showed increased infection after 72 hours, the application of the medicated dressings had still conferred a therapeutic advantage, as indicated by the maintenance of their respective levels of contamination below the clinical infection threshold.

These data demonstrate that application of the cerium nitrate-loaded or cerium nitrate/fentanyl citrate-loaded wound dressings of the present invention is an effective way for maintaining the wound contamination level below the clinical infection threshold for up to 72 hours. Three animals per experimental group were humanely sacrificed 1, 3 or 7 days after a single application of the dressing. Remaining experimental procedures were as described in EXAMPLE 1.

### EXAMPLE 3

### Reference: FIG. 7

This study was designed to compare the bactericidal efficacy of a chlorhexidine-loaded wound dressing of the present invention to that of a commercially available chlorhexidine-loaded wound dressing containing 0.5% chlorhexidine (referred to as COMMERCIAL). Rat wounds were infected as described in EXAMPLE 1. A medicated dressing of the present invention (n=9), a COMMERCIAL dressing (n=9) or a control dressing (i.e., drug-free; n=9) was applied to cover the wound. Layers of the medicated dressings of the present invention were prepared by mixing thoroughly an aqueous solution of the drug with the prepolymer resin. The drug reservoir and wound-contacting layers contained 1% and 0.5% chlorhexidine, respectively. The layers were then cast in sequence to form the final medicated dressing. Three animals per experimental group were humanely sacrificed 1, 3 or 7 days after a single application of the dressing. Remaining experimental procedures were as described in EXAMPLE 1.

All wound dressings of the present invention were removed easily from the wound bed (i.e., no adherence) at all time intervals. In contrast, the commercial dressings had a tendency to adhere to the wounds after the third experimental day. Bacterial counts of untreated wounds remained at or above the clinically accepted threshold of 10⁶ CFU/g over the 7-day study period (FIG. 7). In contrast, wounds covered with the commercial dressing and the chlorhexidine-loaded dressings of the present invention for 24 hours revealed approximately 1.5-log and 3-log reductions, respectively, in the number of bacteria recovered compared to that of the control dressings (FIG. 7). While the reductions in tissue contamination were maintained for 7 days when using the dressing of the present invention, the level of contamination of wounds treated with the commercial dressings was comparable to that of untreated wounds (FIG. 7).

These data suggest that application of the medicated wound dressings of the present invention for up to 7 days is an effective way for preventing the progression of infection in contaminated wounds.

### EXAMPLE 4

### Reference: FIG. 8

The effect of shelf-life on the long-term in vitro bactericidal efficacy of chlorhexidine-loaded wound dressings of the present invention was assessed using a standard zone of inhibition assay. Briefly, 1 cm² wound dressings containing 4% chlorhexidine in both the drug reservoir and hydrogel layer were centered on Mueller-Hinton agar plates seeded with 10⁶ CFU Pseudomonas aeruginosa Following an incubation period of 24 hours at 37° C., the dressing was removed; the zone of inhibition measured in two directions; and, the surface area calculated and corrected for the size of the dressing. Each medicated dressing was then transferred to a freshly seeded Mueller-Hinton agar plate, and the test was repeated daily for up to 8 days. Dressings were tested 13, 33, and 68 days after their manufacture.

FIG. 8 shows that chlorhexidine-loaded wound dressings retained their in vitro bactericidal activity for at least 8 days. Similar results were obtained for dressings containing 4% chlorhexidine in the drug reservoir and 1% chlorhexidine the hydrogel layer (data not shown). Moreover, there was no shelf-life effect on in vitro bactericidal activity.

### EXAMPLE 5

### Reference: FIG. 9

The objective of this study was to compare the effectiveness of various unmedicated wound dressings in cooling human skin. On the experimental day, the skin over the triceps of both arms of eight persons (subjects) was cleansed using alcohol swabs. Two small thermistors were taped 5 cm apart on the skin of each arm, the probes being positioned approximately 10 cm from the tip of the shoulder. The experimental dressings tested were a drug-free wound dressing of the present invention (PI dressing) as well as three commercially available wound-care products comprising a hydrogel sheet, a polyurethane foam dressing, and an amorphous gel. One experimental dressing was centered over each thermistor, and covered with a tape. The experimental dressing was then further secured in place using a 15 cm wide self-adherent non-woven wrap. Temperature recordings were acquired for 6 hours using a small data logger that was worn on a belt.

The effectiveness of the various dressings in cooling the skin is shown in FIG. 9. Within 30 minutes after application of the gel sheet and the polyurethane foam dressing, the skin temperature of the upper arm (Tₛₖᵢₙ) had risen by 1.0° C. and 2.5° C., respectively. While Tₛₖᵢₙ remained constant (30.8° C.) for the remainder of the study in the gel sheet group, it further increased in the polyurethane foam group, reaching a maximum of 33° C. after 60 minutes; and slowly declined for the remainder of the 6-hours study, remaining above the Tₛₖᵢₙ value recorded prior to application of the dressing.

In contrast, Tₛₖᵢₙ markedly dropped (3.0° C.) within 10 minutes of applying the amorphous gel, while Tₛₖᵢₙ under the PI dressing dropped by 1.0° C. However, the cooling effect of the amorphous gel was short-lived, Tₛₖᵢₙ after 30 minutes being comparable to that observed for the PI dressing. While Tₛₖᵢₙ remained constant (29.2° C.) under the PI dressing for most of the 6-hour study, Tₛₖᵢₙ increased steadily under the amorphous gel, reaching a plateau of 30° C. after 90 minutes.

These data demonstrate that the dressing of the present invention can offer a sustained cooling effect for at least 6 hours.

The examples described herein and the disclosure are intended to be illustrative and not exhaustive. These examples and descriptions may well suggest many variations and alternatives to those skilled in the art, all of which may lie within the scope of the present claims.

1. A dressing having a soft polymeric construction comprising a plurality of layers of cellular polymer, at least one layer being a hydrogel surface-contacting layer, and at least one other layer in face-secured relation therewith, being selected from the group consisting of a hydrophilic polymer layer, a drug-containing polymer layer, and a drug-free polymer layer.

2. The dressing as set forth in claim 1 having a surface contacting hydrogel layer as an outermost face layer of said dressing.

3. The dressing as set forth in claim 1, having a drug selected from the group consisting of: broad spectrum antibiotics, antimicrobials, antifungals, antipathogenic peptides, antiseptics, hemostatic agents, local analgesics, central nervous acting agents, wound healing agents, immunomodulatory compounds, and compatible combinations thereof contained in at least one said layer.

4. The dressing as set forth in claim 1, wherein the polymer of said polymer layer is a hydrophilic polyurethane.

5. The dressing as set forth in claim 3, said drug-containing polymer layer comprising a hydrophilic polyurethane prepolymer.

6. The dressing as set forth in claim 5, said hydrophilic polymer face layer being a surface-contacting, drug-containing layer of polyurethane hydrogel.

7. The dressing as set forth in claim 2, said outer layer being positioned on the side of said dressing remote from a contact surface, being adjacent said drug-containing layer.

8. The dressing as set forth in claim 1, wherein said layers are secured in mutual face-contacting relation.

9. The dressing as set forth in claim 1, including a release sheet secured in protective relation over a face surface of the dressing.

10. The dressing as set forth in claim 1, wherein at least two said layers in face-adjoined relation include a flow sensitive transfer means in interposed relation at said adjoined face, to control the transfer of fluid between said layers, whereby consequent deformation of a said layer is substantially controlled.

11. The dressing as set forth in claim 1, one said layer being a hydrophilic polyurethane incorporating at least one agent selected from the group consisting of surfactant agents and super-absorbant agents.

12. The dressing as set forth in claim 11, wherein said super-absorbent agent is selected from the group consisting of alginates and chitosan.

13. The dressing as set forth in claim 1, wherein one said layer has a plurality of channels within said layer, to promote dispersion of fluid within the layer.

14. The dressing as set forth in claim 13, wherein one layer is an outer, surface-contacting layer of said dressing.

15. The dressing as set forth in claim 1 having an outer, surface-contacting hydrogel layer, wherein the surface of said outer layer is discontinuous, to provide fluid distribution channels.

16. The dressing as set forth in claim 13, wherein one said layer consists of a plurality of separate layer portions in mutually spaced relation, being sprayed in adhering relation to an adjacent said layer.

17. The method of manufacturing a dressing as set forth in claim 1, comprising the steps of:
a. mixing the components of the surface-contacting layer together to form a standardized aerated mixture;
b. spreading the aerated mixture onto a smooth support surface to form a mixture sheet of pre-determined thickness;
c. mixing the components of said drug containing layer to form a standardized aerated mixture; and
d. spreading the aerated mixture of step c) on top of the mixture sheet of step b) before the mixture of step b) is fully cured, and allowing both layers to cure simultaneously.

18. The method as set forth in claim 17, wherein said drug in said drug reservoir layer is mixed with the components of said drug-reservoir layer, in carrying out said step c)

19. The method as set forth in claim 17, wherein said drug in said surface-contacting layer is mixed with the components of said surface-contacting layer, in carrying out said step a).

20. The method as set forth in claim 17, further including the steps of: mixing the components of a second surface-contacting layer together to form a standardized aerated mixture; and, spreading said aerated mixture on top of said mixture of said step c).

21. The method as set forth in claim 17, wherein said drug is incorporated into said dressing after said layers have cured, by immersing the dressing in an aqueous solution containing said drug.

22. The method as set forth in claim 17, wherein said drug is absorbed under pressure into said dressing subsequent to the curing of said layers, said dressing layers being immersed in a solution containing said drug.

23. A method for manufacturing the dressing of claim 1, said method comprising:
a. mixing the components of said surface-contacting layer together to form a standardized aerated mixture;
b. spreading the aerated mixture of step a) onto a smooth support to form a sheet of pre-determined thickness, and allowing the sheet to fully cure;
c. mixing the components of said drug reservoir layer to form a standardized aerated mixture;
d. spreading the aerated mixture of step c) onto a smooth support to form a sheet of pre-determined thickness, and allowing the sheet to fully cure; and
e. sealing the sheet obtained in step b) to the sheet obtained in step d).

24. The method as set forth in claim 23, wherein said sealing step e) is selected from the process group consisting of heat sealing, radio frequency welding, discontinuous adhesive, and ultrasonic welding.

25. The method of manufacturing a dressing, comprising the steps of:
a. mixing the components of the surface-contacting layer together to form a standardized aerated mixture;
b. spreading the aerated mixture onto a smooth support to form a sheet of predetermined thickness;
c. mixing the components of said drug containing layer to form a standardized aerated mixture; and
d. spreading the aerated mixture of step c) on top of the mixure of step b) before the mixture of step b) is fully cured, and allowing both layers to cure simultaneously.

26. The method as set forth in claim 25, wherein said drug in said drug reservoir layer is mixed with the components of said drug-reservoir layer, in carrying out said step c).

27. The method as set forth in claim 25, wherein said drug in said surface-contacting layer is mixed with the components of said surface-contacting layer, in carrying out said step a).

28. The method as set forth in claim 25, further including the steps of: mixing the components of a second surface-contacting layer together to form a standardized aerated mixture; and spreading said aerated mixture on top of said mixture of said step c).

29. The method as set forth in claim 25, wherein said drug is incorporated into said dressing after said layers have cured, by immersing the dressing in an aqueous solution containing said drug.

30. The method as set forth in claim 25, wherein said drug is absorbed under pressure into said dressing subsequent to the curing of said layers, said dressing layers being immersed in an aqueous solution containing said drug.

31. A method for manufacturing a dressing, comprising: the steps of:
a. mixing the components of said surface-contacting layer together to form a standardized aerated mixture;
b. spreading the aerated mixture of step a) onto a smooth support to form a sheet of pre-determined thickness, and allowing the sheet to fully cure;
c. mixing the components of said drug reservoir layer to form a standardized aerated mixture;
d. spreading the aerated mixture of step c) onto a smooth support to form a sheet of pre-determined thickness, and allowing the sheet to fully cure; and
e. sealing the sheet obtained in step b) to the sheet obtained in step d).

32. The method as set forth in claim 31, wherein said sealing step e) is selected from the process group consisting of heat sealing, radio frequency welding, discontinuous adhesive, and ultrasonic welding.

33. A method of delivering a drug selected from a predetermined group to an external area of damaged tissue of a patient, consisting of the step of applying a dressing as set forth in claim 1 to said area.

33. A method of delivering a drug selected from a predetermined group to an internal area of damaged tissue of a patient, consisting of the step of applying a dressing as set forth in claim 1 to said area.
